(19) United States
(12) Patent Application Publication
(10) Pub. No.: US 2020/0367491 A1
(43) Pub. Date: Nov. 26, 2020

(54) LIPOSOMAL FORMULATIONS AND METHODS OF USING SAME IN AGRICULTURE

(71) Applicant: TECHNION RESEARCH AND DEVELOPMENT FOUNDATION LTD., Haifa (IL)

(72) Inventors: Avi SCHROEDER, Binyamina (IL); Avishai KARNY, Haifa (IL)

(73) Assignee: TECHNION RESEARCH AND DEVELOPMENT FOUNDATION LTD., Haifa (IL)

(21) Appl. No.: 16/987,282

(22) Filed: Aug. 6, 2020

Related U.S. Application Data
(63) Continuation of application No. 16/082,751, filed on Sep. 6, 2018.
(60) Provisional application No. 62/305,629, filed on Mar. 9, 2016.

(51) Int. Cl. A01N 25/28; A01N 25/30; A61K 9/127; A01N 57/20

(52) U.S. Cl. CPC A01N 25/28; A01N 25/30; A01N 43/16; A01N 57/20; A61K 9/1272

(57) ABSTRACT

Provided is a formulation including an agriculturally acceptable carrier and liposomes including a lipid membrane and an intraliposomal aqueous core. The liposome has a diameter in the range of between 100 nm to 300 nm and the lipid membrane includes at least one liposome forming phospholipid. The lipid membrane may include two or more phospholipids. At least one of the two or more phospholipids may be a liposome forming lipid. Further, at least one of the two or more phospholipids may be characterized by one or more of the following features: (a) it has an unsaturated lipid tail; (b) it includes a polar head group; (c) it includes an acidic head group. Further provided are methods of using the formulation for agricultural treatment and kits for preparing and using such formulations.

### TECHNOLOGICAL FIELD

The present disclosure relates to agriculture and specifically to formulations for delivery of agriculturally active agents.

### BACKGROUND ART

References considered to be relevant as background to the presently disclosed subject matter are listed below:
U.S. Pat. No. 4,394,149
U.S. Pat. No. 5,958,463
U.S. Pat. No. 6,165,500
International patent application publication No. WO 13/192190

Acknowledgement of the above references herein is not to be inferred as meaning that these are in any way relevant to the patentability of the presently disclosed subject matter.

### BACKGROUND

In agriculture delivery of active agents is at times hampered by the presence of the cuticle layer that prevents the agent from penetration into the plant's vascular system and delivery of same to the various plant parts.

U.S. Pat. No. 4,394,149 describes liposomes encapsulating biologically active material. The liposomes are formed by mixing lipid or organic solvent with aqueous solution of the biologically active material, emulsifying the mixture, removing the solvent and suspending the gel in water.

U.S. Pat. No. 5,958,463 describes a method for preparing boron containing liposomes for agricultural use. The method involves mixing lecithin with organic solvent in specific proportions. After allowing the mixture to settle the top layer is saved while the bottom layer is discarded. Next the active agent is added to form a concentrate. When the concentrate is added to water the vesicle is formed.

U.S. Pat. No. 6,165,500 describes the preparation of liposomes that comprise a lipid and surfactant (referred to as transfersomes) for transporting medical agent through membranes. The transfersomes were shown to penetrate into the surface of leaves which resulted in a slightly reddish appurtenance at the surface of the leaves.

Finally, International patent application publication No. WO 13/192190 describes liposomal formulation for agricultural use, that comprise as active ingredient pesticides, nematicides, or herbicides. The formulation is applied to the soil or to the plant media.

### General Description

The present disclosure provides, in accordance with a first of its aspects, a formulation comprising (i) liposomes comprising a lipid membrane and an intraliposomal aqueous core, wherein the liposome has a diameter in the range of between 100 nm to 300 nm; and the lipid membrane comprises at least one liposome forming phospholipid; and (ii) an agriculturally acceptable carrier.

In some embodiments, the present disclosure provides a formulation comprising:
(i) liposomes comprising a lipid membrane and an intraliposomal aqueous core, wherein,
   the liposome has a diameter in the range of between 100 nm to 300 nm
   the lipid membrane comprises two or more phospholipids,
   at least one of said two or more phospholipids is a liposome forming lipid, and
   at least one of said two or more phospholipids is characterized by one or more of the following features: (a) it has an unsaturated lipid tail; (b) it comprises a polar head group; (c) it comprises an acidic head group; and
(ii) an agriculturally acceptable carrier.

In accordance with a second aspect, the present disclosure provides a method of treatment, comprising applying the formulation disclosed herein. In some embodiments, the method is for treating a plant and comprises applying to a surface of a plant part a formulation as disclosed herein.

Also disclosed herein is the use of a formulation as disclosed herein for agriculture.

In accordance with a third aspect, the present disclosure provides a kit comprising (a) an agriculturally acceptable carrier; (b) liposomes or liposome forming lipids as defined herein; and (c) instructions for use of the carrier and liposomes for treating a plant.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B present the effect of Liposome A size on % Gd penetration (FIG. 1A) and distribution to different plant organs (FIG. 1B).

FIGS. 2A-2B present % Gd penetration (FIG. 2A) and total Gd distribution in the plant (FIG. 2B) when encapsulated in Liposome A, Liposome B or in free form, yet in the presence of 0.1% surfactant.

FIGS. 3A-3B present the effect of cholesterol in Liposome A on % Gd penetration (FIG. 3A) and Gd distribution to the plant organs (FIG. 3B).

FIGS. 4A-4B present the effect of PEG-DSPE on % Gd penetration (FIG. 4A) and Gd distribution in the plant organs (FIG. 4B) when in Liposome A.

FIGS. 5A-5B present the effect of chain length of the phospholipid, namely, HSPC, DPPC or DMPC on total Gd distribution (FIG. 5A) and Gd distribution to different plant organs (FIG. 5B) when in Liposome A.

FIGS. 6A-6C present the effect of the presence of a cationic lipid, DOTAP on total Gd distribution (FIG. 6A) and Gd distribution to different plant organs (FIG. 6B) when in Liposome A, as well as on the total Gd distribution (FIG. 6C) when in the formulation of Liposome B.

FIGS. 7A-7C present the effect of 10% tocopherol on total Gd distribution (FIG. 7A) and Gd distribution to different plant organs (FIG. 7B) when of Liposome A, or on total Gd distribution when in the formulation of Liposome B (FIG. 7C).

FIGS. 8A-8H are confocal microscopy images showing intracellular uptake of liposomal Fluorescein and release in the roots 24 hr (FIG. 8A), 48 hr (FIG. 8B), 72 hr (FIG. 8C) and 96 hr (FIG. 8D) after foliar application of Fluorescein-encapsulated Liposome A or after 72 hr when encapsulated in Liposome B (FIG. 8E), and cellular uptake and release in protoplasts of adjacent leaves after 24 hr (FIG. 8F), 48 hr (FIG. 8G), 72 hr (FIG. 8H).

FIG. 9 is a graph showing lateral translocation of EuC13 when in formulation of Liposome A, applied by leaf submerging of a single mature vine leaf.

FIG. 10 is an image showing herbicidal activity of Glufosinate applied by smearing onto a single leaf of three plants of Eleusina Indica (application leaves marked by arrows), the glufosinate being applied as part of a commercial product Faster™ (Tapazol, 200 g/L glufosinate ammonium) (left plant) at the recommended rate, within Liposome B (center plant) at 65% of recommended rate, both being compared to untreated plant (right plant).

FIGS. 11A-11E is an image showing herbicidal activity after 22 days (FIGS. 11A-11C) or 35 days (FIG. 11D-11E) of spraying Glufosinate on the entire foliage of cotton plants, the glufosinate being applied at 1/16 the recommended dose (0.375 mg/ml) (FIG. 11B, FIG. 11D, respectivelty), as compared to the same amount within Liposome B (0.35 mg/ml, FIG. 11C and FIG. 11E, repsectively), and as compared to untreated plant (FIG. 11A).

FIGS. 12A-12F are images showing Mg deficiency correction in 3^{rd} and 4^{th} leaf (FIGS. 12A-12C and FIGS. 12D-12F, respectively) by foliar application on the topmost leaf of Mg encapsulated in Liposome A (FIG. 12C, FIG. 12F), as compared to MgSO₄, which is a standard Mg formulation common for foliar application in orchards (FIG. 12B, FIG. 12E), or untreated plant (FIG. 12A, FIG. 12D)

FIGS. 13A-13D are images showing Mg and Fe deficiency correction in 3^{rd} and 4^{th} leaf and whole plant (FIGS. 13A-13C and FIGS. 13D-13F, 13G-13I, respectively) by foliar application on the topmost leaf of Mg and Fe encapsulated in Liposome A (FIG. 13C, FIG. 13F), as compared to a mixture of standard Mg and Fe formulations (MgSO₄ and Sequestrene™ (chelated Fe, BASF) (FIG. 13B, FIG. 13E, FIG. 13G), or untreated plant (FIG. 13A, FIG. 13D, 13I).

FIG. 14A-14D show Fe deficiency correction by foliar application on the lowest leaf of Sequestrene™ (chelated Fe, BASF) (FIG. 14A) or non-chelated Fe (FIG. 14C), as compared to Sequestrene™ in Liposome A (FIG. 14B) and non-chelated Fe in Liposome A (FIG. 14D).

### DETAILED DESCRIPTION OF EMBODIMENTS

The present disclosure is based on the development of a liposomal formulation, applied onto leaves of a plant, that was effective in distributing an agent encapsulated within the intraliposomal core of the liposome into various plant parts, including the apical shoot, stem and roots.

Thus, the present disclosure provides, in accordance with its first aspect, a formulation comprising (i) liposomes comprising a lipid membrane and an intraliposomal aqueous core, wherein the liposome has a diameter in the range of between 100 nm to 300 nm; and the lipid membrane comprises at least one liposome forming phospholipid; and (ii) an agriculturally acceptable carrier.

In accordance with some embodiments, the present disclosure provides, a liposomal formulation comprising within a carrier suitable for agricultural use, liposomes comprising a lipid membrane and an intraliposomal aqueous core, wherein
the liposomes have a diameter in the range of between 100 nm to 300 nm,
the lipid membrane comprise two or more phospholipids,
at least one of said two or more phospholipids is a liposome forming lipid, and
at least one of said two or more phospholipids is characterized by one or more of the following features: (a) it has an unsaturated lipid tail; (b) it comprise a polar head group; (c) it comprises an acidic head group.

Liposomes are sealed sacs in the micron and submicron range dispersed in an aqueous environment in which one or more bilayers (lamellae) separate the external aqueous phase from the internal aqueous phase. The bilayer is composed of amphiphiles, the latter having defined polar and apolar regions. When amphiphiles are present in an aqueous phase, they self-aggregate such that their hydrophilic moiety faces the aqueous phase, while their hydrophobic domain is “protected” from the aqueous phase.

As a prerequisite in order to form liposomes, amphiphiles must be organized in a lamellar phase. However, the formation of lamellar phases is not sufficient to lead to liposome formation [Seddon, J. M., Biochemistry, 29(34):7997-8002, (1990)]. Liposome formation also requires the ability of the lamellae to close up on themselves to form vesicles.

Various approaches were proposed to classify amphiphiles into sub-groups. One approach is based on geometric and energetic parameters of amphiphiles. According to this approach proposed by Israelachvili and co-workers [Israelachvili, J, Physical principles of membrane organization, Q. Rev Biophys, 13(2):121-200 (1980), Lichtenberg and Barenholz, In Methods of Biochemical Analysis, D. Glick (Ed), 33:337-462, 1988; Kumar, Biophys J., 88:444-448, (1991)] amphiphiles are defined by a packing parameter (PP), which is the ratio between the cross sectional areas of the hydrophobic and hydrophilic regions.
Amphiphiles with a packing parameter of ˜1.0 (cylinder-like molecules) form a lamellar phase and have a potential to form liposomes;
Amphiphiles with a larger than 1.0 packing parameter (inverted cone-shaped molecules) tend to form hexagonal type II (inverted hexagonal) phases. Such amphiphiles when having very small headgroup disperse hardly and in some cases do not even swell in the aqueous phase;
Amphiphiles with a smaller packing parameter of ≥⅔ (cone-shaped molecules) will self-aggregate as micelles. Examples of micelle forming amphiphiles which self-aggregate include phospholipids with short hydrocarbon chains, or lipids with long hydrocarbon chains (<10 carbon atoms), but with large, bulky polar head-groups (e.g. gangliosides and lipopolymers composed of a lipid to which a polyethylene glycol (PEG) moiety (≥750 Da) is covalently attached) [Israelachvili, J. N., In Intermolecular and surface forces, 2^{nd} Ed. Academic Press, pp 341-365, (1992); Lichtenberg and Barenholz, Supra, (1988); Barenholz and Cevc, In Physical Chemistry of Biological Surfaces, Marcel Dekker, N.Y., pp 171-241, (2000)].

In the context of the present disclosure, the liposomes are used as a carrier for agriculturally beneficial agents and to this end, various types of liposomes can be used. Specifically, yet without being limited thereto, the liposomes of the disclosed formulation can be any one or combination of vesicles selected from the group consisting of small unilamellar vesicles (SUV), large unilamellar vesicles (LUV), multilamellar vesicles (MLV), multivesicular vesicles (MVV), large multivesicular vesicles (LMVV, also referred to, at times, by the term giant multivesicular vesicles, “GMV”), oligolamellar vesicles (OLV), and others.

In some embodiments, the liposomes are large unilameller vesicles (LUV).

It has been found by the inventors that there is a significance, for the purpose of, inter alia, liposome penetration and/or distribution within the plant, to use a liposomal population having an average diameter in the range of 100 to 300 nm. In some embodiments, the liposomes have an average diameter in the range of 100 nm to 250 nm, at times, in the range of 110 nm to 230 nm, at times, in the range of 120 nm to 220 nm, at times, at least 100 nm but no more than 200 nm.

The lipid membrane comprises two or more phospholipids, at least one of which is a liposome forming lipid. It is noted in this connection that the amount of phospholipids in the liposome can be determined as organic phosphorous by the modified Bartlett method [Shmeeda H, Even-Chen S, Honen R, Cohen R, Weintraub C, Barenholz Y. 2003. Enzymatic assays for quality control and pharmacokinetics of liposome formulations: comparison with nonenzymatic conventional methodologies. Methods Enzymol 367:272-92].

As used herein, the “Liposome forming lipids” are firstly phospholipids which when dispersed in aqueous media by itself at a temperature above their solid ordered to liquid disordered phase transition temperature (Tₘ, the temperature in which the maximal change in heat capacity occurs during the phase transition) will form stable liposomes.

In some embodiments, the phospholipids are selected from glycerophospholipids and sphingomyelins. The glycerophospholipids have a glycerol backbone wherein at least one, preferably two, of the hydroxyl groups at the head group is substituted by one or two hydrocarbon tails (chains), typically, an acyl, alkyl or alkenyl tails, and the third hydroxyl group is substituted by a phosphate (phosphatidic acid) or a phospho-ester such as phosphocholine group (as exemplified in phosphatidylcholine), being the polar head group of the glycerophospholipid or combination of any of the above, and/or derivatives of same and may contain a chemically reactive group (such as an amine, acid, ester, aldehyde or alcohol). The sphingomyelins consists of a ceramide (N-acyl sphingosine) unit having a phosphocholine moiety attached to position 1 as the polar head group. The term “sphingomyelin” or “SPM” as used herein denotes any N-acetyl sphingosine conjugated to a phosphocholine group, the later forming the polar head group of the sphingomyelin (N-acyl sphingosyl phospholcholines). The acyl chain bound to the primary amino group of the sphingosine (to form the ceramide) may be saturated or unsaturated, branched or unbranded.

In some embodiments, at least one of the liposome forming lipid is a phospholipid having one or two C14 to C24 hydrocarbon tails, typically, acyl, alkyl or alkenyl chain) and have varying degrees of unsaturation, from being fully saturated to being fully, partially or non-hydrogenated lipids (the level of saturation may affect rigidity of the liposome thus formed (typically liposomes formed from lipids with saturated chains are more rigid than liposomes formed from lipids of same chain length in which there are unsaturated chains, especially having cis double bonds).

Further, the lipid membrane may be of natural source (e.g. naturally occurring phospholipids), semi-synthetic or fully synthetic lipid, as well as electrically neutral, negatively or positively charged.

Examples of liposome forming glycerophospholipids include, without being limited thereto, phosphatidylglycerols (PG) including dimyristoyl phosphatidylglycerol (DMPG); phosphatidylcholine (PC), including egg yolk phosphatidylcholine, soybean PC, sunflower PC, rapeseed PC, krill PC, canola PC, flax seed lecithin, wheat lecithin, dimyristoyl phosphatidylcholine (DMPC, Tm 24° C.), 1-palmitoyl-2-oleoylphosphatidyl choline (POPC), hydrogenated soy phosphatidylcholine (HSPC, Tm 65° C.), distearoylphosphatidylcholine (DSPC, Tm 55° C.); di-lauroyl-sn-glycero-2phosphocholine (DLPC); 1,2-dipalmitoyl-sn-glycero-3-phosphocholine (DPPC, Tm 41° C.); 1,2-dinonadecanoyl-sn-glycero-3-phosphocholine; 1,2-diarachidoyl-sn-glycero-3-phosphocholine (DBPC); 1,2-dihenarachidoyl-sn-glycero-3-phosphocholine; 1,2-dibehenoyl-sn-glycero-3-phosphocholine 1,2-ditricosanoyl-sn-glycero-3-phosphocholine 1,2-dilignoceroyl-sn-glycero-3-phosphocholine; 1-myristoyl-2-stearoyl-sn-glycero-3-phosphocholine; 1-palmitoyl-2-stearoyl-sn-glycero-3-phosphocholine (PSPC); 1-stearoyl-2-palmitoyl-sn-glycero-3-phosphocholine (SPPC); 1,2-di-oleoyl-sn-glycero-3-phosphocholine (DOPC-1.7° C.); phosphatidic acid (PA), phosphatidylinositol (PI), phosphatidylserine (PS), phosphatidylethanolamine (PE).

In some embodiments, the at least one liposome forming lipid has a choline head group.

In some embodiments, the at least one liposome forming lipid is a phosphatidylcholine (PC) carrying one or two saturated or unsaturated C14 to C24 hydrocarbon tails, or at times or two saturated or unsaturated C14 to C20 hydrocarbon tails, or at times or two saturated or unsaturated C16 to C20 hydrocarbon tails, or at times one or two saturated or unsaturated C16 or C18 hydrocarbon tails.

In some embodiments, the lipid membrane comprises a combination of PC's carrying at least one hydrocarbon tail selected from the group consisting of C16:0, C18:0, C18:1, C18:2, and C18:3.

In some embodiments, the lipid membrane comprises at least one unsaturated C16 or C18 PC.

In some embodiments, the lipid membrane comprises a mole ratio between saturated and non-saturated liposome forming lipids of between 10%:90% to 90%:10%, at times, a mole ratio of between 20%:80% to 80%:20%, at times, a mole ratio between 30%:70% to 70%:30%, at times, a mole ratio between 20%:80% to 50%:50%, at times, a mole ratio of between 20:80 to 40%:60%.

In some embodiments, at least one of said two or more phospholipids comprise at least one unsaturated hydrocarbon tail, namely, at least one double bond in the hydrocarbon chain. At times, the unsaturated hydrocarbon chain can comprise two, three, four or more double bonds.

In some embodiments, at least one of said two or more phospholipids is a lipid comprising a polar head group.

In the context of the present disclosure, when referring to a polar head group it is to be understood as one encompassing an alcohol moiety.

In some embodiments, the polar head group is one comprising a serine moiety.

In some embodiments, the polar head group is one comprising a choline moiety.

In some embodiments, the polar head group is one comprising ethanolamine.

In some embodiments, the polar head group is one comprising glycerol.

In some embodiments, at least one of said two or more phospholipids comprise a polar inositol head group. In some embodiments, the phospholipid comprising an inositol head group is selected from the group consisting of phospatidylinositol (PI), PI(4)P, PI(3)P, PI(3,4,5)P3, PI(4,5)P2, PI(3,5)P2, PI(3,4)P2.

In some embodiments, at least one of said two or more phospholipids has an acidic head group.

In some embodiments, when referring to an “acidic head group” it is to be understood as encompassing a moiety selected from the group consisting of glycerol, hydroxyl, carboxyl, amine, and phosphoric group.

Examples of the acidic phospholipids include natural or synthetic lipid selected from phosphatidylglycerols (PGs) such as dilauroylphosphatidylglycerol (DLPG) dimyristoylphosphatidylglycerol (DMPG) dipalmitoylphosphatidylglycerol (DPPG), distearoylphosphatidylglycerol (DSPG) dioleoylphosphatidylglycerol (DOPG), egg yolk phosphatidylglycerol (egg yolk PG), hydrogenated egg yolk phosphatidylglycerol; phosphatidylinositols (PIs) such as phosphatidylinositol, dimyristoylphosphatidylinositol, dipalmitoylphosphatidylinositol (DPPI), distearoylphosphatidylinositol (DSPI), dioleoylphosphatidylinositol (DOPI), soybean phosphatidylinositol (soybean PI), hydrogenated soybean phosphatidylinositol, phosphoinositides, sphingomyelin and phosphatidic acid. Each of these acidic phospholipids can be used alone or in combination of two or more.

In some embodiments, the acidic head group comprises a phosphatidic acid.

In some embodiments, the liposomes in the disclosed formulation comprise within the lipid membrane at least one non-liposome forming lipid.

When referring to a non-liposome forming lipid it is to be understood as referring to a lipid that does not spontaneously form into a vesicle when brought into an aqueous medium.

There are various types of lipids that do not spontaneously vesiculate and yet are used or can be incorporated into vesicles. These include, for example, sterols, saponins, sphingolipids, e.g. sphingomyelin, lipoproteins, e.g. PEG-DSPE, etc.

Such additional lipids can be used to affect any one of the stability, surface charge and membrane fluidity, as well as assist in the loading of active agents into the liposomes.

In some embodiments, the non-liposome forming lipid is a sterol.

A non-limiting list of sterols that can be part of the lipid membrane of the liposomes includes β-sitosterol, β-sitostanol, stigmasterol, stigmastanol, campesterol, campestanol, ergosterol, avenasterol, brassicasterol, fucosterol, cholesterol (CHOL), cholesteryl hemisuccinate, and cholesteryl sulfate.

In some embodiments, the sterol is a plant derived sterol, namely, a phytosterol. In accordance with this embodiment, the sterol is selected from the group consisting of β-sitosterol, β-sitostanol, stigmasterol, stigmastanol, campesterol, campestanol, ergosterol, avenasterol, brassicasterol and any combination of two or more of these sterols.

In some further embodiments, the lipid membrane comprises one or more phytosterols selected from the group consisting of β-sitosterol, stigmasterol, and ergosterol.

Another non-liposome forming lipid that can form part of the lipid membrane is a hydrophobic aglycone or a saponin. Structurally, saponins contain a hydrophobic aglycone and a hydrophilic glycoside (sugar) head group.

Saponins can be used as an alternative to sterols or they can be used in combination with sterols. In some embodiments, the lipid membrane comprises a combination of at least one sterol and at least one saponin.

When the lipid membrane comprise a combination of one or more sterol and one or more saponins, the mole % ratio between the phospholipids in the lipid membrane and the sterols and saponins in the lipid membrane is between 20 mol %:80 mol % to 80 mol %:20 mol %. At times, the mole ratio between the phospholipids and the sterols and saponins is between 40 mol %:60 mol % to 60 mol %:40 mol %. At times, the mole ratio between the phospholipids and the sterols and saponins is between 50 mol %:50 mol % to 80 mol %:20 mol %.

In some embodiments, the saponin is selected from the group consisting of dammaranes, tirucallanes, lupanes, hopanes, oleananes, taraxasteranes, ursanes, cycloartanes, lanostanes, cucurbitanes, solanine, solanidine, tomatine, chaconine, tomatidine and steroids, with or without a linked sugar moiety.

In some embodiments, the saponin is selected from the group consisting of solanine, solanidine, tomatine, chaconine, tomatidine and any combination of same.

The ratio between the liposome forming lipid and the non-liposome forming lipid can vary depending on the desired effect of the non-liposome forming lipids on the membrane. At times, the lipid membrane comprises a liposome forming lipid to non-liposome forming lipid mole ratio of between 20%:80% to 80%:20%, at times, a mole ratio of between 30%:70% to 70%:30%, at times, a mole ratio of between 40%:60% to 60%: 40%.

In some embodiments, the lipid membrane comprises at least one cationic lipid (monocationic or polycationic lipids). Cationic lipids typically consist of a lipophilic moiety, such as a sterol or the same glycerol backbone to which two acyl or two alkyl, or one acyl and one alkyl chain contribute the hydrophobic region of the amphipathic molecule, to form a lipid having an overall net positive charge.

Monocationic lipids may include, for example, 1,2-dimyristoyl-3-trimethylammonium propane (DMTAP) 1,2-dioleyloxy-3-(trimethylamino) propane (DOTAP); N-[1-(2,3-ditetradecyloxy)propyl]-N,N-dimethyl-N-hydroxyethylammonium bromide (DMRIE); N-[1-(2,3, -dioleyloxy)propyl]-N,N-dimethyl-N-hydroxy ethyl-ammonium bromide (DORIE); N-[1-(2,3-dioleyloxy) propyl]-N,N,N-trimethylammonium chloride (DOTMA); 3β-[N—(N′,N′-dimethylaminoethane) carbamoly] cholesterol hydrochloride (DC-Chol); and dimethyl-dioctadecylammonium (bromide salt, DDAB), N1-[2-((1S)-1-[(3-aminopropyl)amino]-4-[di(3-amino-propyl)amino] butylcarboxamido)ethyl]-3,4-di[oleyloxy]-benzamide (MLV), 1,2-di-O-octadecenyl-3-trimethylammonium propane (chloride salt) (DOTMA), Ethyl PC, 1,2-dioleoyl-3-dimethylammonium-propane (DODAP), N-(4-carboxybenzyl)-N,N-dimethyl-2,3-bis(oleoyloxy)propan-1-aminium (DOBAQ)

Polycationic lipids, due to their large polycationic head group may, at times, be considered as non-liposome forming lipids. Such lipids, when mixed with other lipids such as sterols and saponins together with liposome forming phospholipids at suitable mole ratio will be incorporated into the lipid membrane of the liposomes. The polycationic lipids include a similar lipophilic moiety as with the mono cationic lipids, to which polycationic head groups are covalently attached such as the polyalkyamines spermine or spermidine. The polycationic lipids include, without being limited thereto, N-[2-[[2,5-bis [3-aminopropyl)amino]-1-oxopentyl] amino] ethyl]-N,N-dimethyl-2, 3-bis[(1-oxo-9-octadecenyl)oxy]-1-propanaminium (DOSPA), and ceramide carbamoyl spermine (CCS). The cationic lipids may form part of a derivatized phospholipids such as the neutral lipid dioleoylphosphatidyl ethanolamine (DOPE) derivatized with polylysine to form a cationic lipopolymer.

The liposomes may further comprise lipopolymers. The term “lipopolymer” is used herein to denote a lipid substance modified by inclusion in its polar head group a hydrophilic polymer. The polymer head group of a lipopolymer is typically water-soluble. Typically, the hydrophilic polymer has a molecular weight equal or above 750 Da. There are numerous polymers which may be attached to lipids to form such lipopolymers, such as, without being limited thereto, polyethylene glycol (PEG), polysialic acid, polylactic (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers. The lipids derivatized into lipopolymers may be neutral, negatively charged, as well as positively charged.

The most commonly used and commercially available lipids derivatized into lipopolymers are those based on phosphatidyl ethanolamine (PE), usually, distearoylphosphatidylethanolamine (DSPE).

One particular family of lipopolymers that can be employed according to the present disclosure are the monomethylated PEG attached to DSPE (with different lengths of PEG chains, in which the PEG polymer is linked to the lipid via a carbamate linkage resulting in a negatively charged lipopolymer, or the neutral methyl polyethyleneglycol distearoylglycerol (mPEG-DSG) and the neutral methyl poly ethyleneglycoloxy carbonyl-3-amino-1,2-propanediol distearoylester (mPEG-DS) [Garbuzenko O. et al., Langmuir. 21:2560-2568 (2005)]. Another lipopolymer is the phosphatidic acid PEG (PA-PEG).

The PEG moiety can have a molecular weight of the head group from about 750 Da to about 20,000 Da, at times, from about 750 Da to about 12,000 Da and typically between about 1,000 Da to about 5,000 Da. While the lipids modified into lipopolymers may be neutral, negatively charged, as well positively charged, i.e. there is not restriction to a specific (or no) charge. For example, the neutral distearoyl glycerol and the negatively charged distearoyl phosphatidylethanolamine, both covalently attached to methoxy poly(ethylene glycol) (mPEG or PEG) of Mw 750, 2000, 5000, or 12000 [Priev A, et al. Langmuir 18, 612-617 (2002); Garbuzenko O., Chem Phys Lipids 135, 117-129(2005); M. C. Woodle and DD Lasic Biochim. Biohys. Acta, 113, 171-199. 1992].

The most commonly used and commercially available lipids derivatized into lipopolymers are those based on phosphatidyl ethanolamine (PE), usually, distearylphosphatidylethanolamine (DSPE). A specific family of lipopolymers employed by the invention include methoxy PEG-DSPE (with different lengths of PEG chains) in which the PEG polymer is linked to the DSPE primary amino group via a carbamate linkage. The PEG moiety preferably has a molecular weight of the head group is from about 750 Da to about 20,000 Da. More preferably, the molecular weight is from about 750 Da to about 12,000 Da and most preferably between about 1,000 Da to about 5,000 Da. One specific PEG-DSPE that can be employed herein is that wherein PEG has a molecular weight of 2000 Da, designated herein ²⁰⁰⁰PEG-DSPE or ^{2k}PEG-DSPE [M. C. Woodle and DD Lasic Biochim. Biohys. Acta, 113, 171-199. 1992].

In some embodiments, the liposomes contain up to 5 mole % lipopolymer. In some embodiments, the liposome is either lipopolymer free or contains between 0.1 mole % to 5 mole %, at times, between 0.5 mole % to 4 mole %, at times between 1 mole % to 3 mole %.

In some embodiments, the liposome contains at least one anti-oxidant. In some embodiments, the anti-oxidant is within the lipid membrane.

In some embodiments, the anti-oxidant is selected from the group consisting of α-tocopherol, tocotrienols, tocopherol succinate, tocopherol acetate, ascorbyl palmitate, coenzyme Q10 (ubiquinone), vitamin A, bioflavonoids, carotenoids, sodium escorbate, glutathione.

In some embodiments, the anti-oxidant is α-tocopherol.

In some embodiments, the liposome comprise a targeting moiety exposed at the external surface of the liposomes. In some embodiments, the targeting moiety is a low molecular weight compound, a protein, a peptide or a glycoprotein linked embedded to at least the outer surface of the liposomes.

In some embodiments, the liposomes can comprise, embedded in the lipid membrane, a protein that can facilitate specific plant organ targeting or penetration.

In some embodiments, the liposome can comprise other hydrophobic and/or other lipids or combination of lipids such as glycosphingolipids (i.e., gangliosides), and phosphatidyl ethanolamines (PE). Such groups would typically have a functional group extending from the liposome membrane, the exposed groups may then be used, for example, as a targeting moiety. Examples of such exposed groups may include, without being limited thereto, sugars (glycolipid), polymers (lipopolymer), proteins (lipoprotein).

In some embodiments, the lipid membrane comprises lipids that are essentially all from natural source. In some embodiments, the lipid membrane comprises lipids that are all from plant source.

When referring to a lipid or lipids from plant source it is to be understood that the lipid(s) is isolated from a plant or from a part of a plant (e.g. the seeds) such that when applied onto a plant as part of the formulation disclosed herein, no plant hyper sensitive response is launched.

In some embodiments, natural/plant derived lipids can be obtained from vegetable sources like, e.g., seed oil (from soybeans, rape (canola), wheat germ, sunflower, flax, cotton, corn, coconut, arachis, sesame), pulp oil (palm, olive, avocado pulp), desert shrub, tobacco, bean, and carrot. These raw materials are world-wide produced at very large scale. Natural phospholipids may be further converted to saturated phospholipids by means of hydrogenation or further treated with enzymes to, e.g., remove partially fatty acids (e.g. using phospholipase A2) or to convert a polar head group (e.g. using phospholipase D). The saturated phospholipids are considered as natural phospholipids because the resulting saturated lipids are also occurring in nature (i.e., natural identical).

In accordance with some embodiments, the lipid membrane comprises plant derived phospholipids comprising lecithin or portion thereof. Lecithin is described in the United States Pharmacopoeia (USP) as a complex mixture of acetone-insoluble phosphatides, which consists chiefly of PC, PE, phosphatidylserine, and phosphatidylinositol, combined with various amounts of other substances such as triglycerides, fatty acids, and carbohydrates, as separated from the crude vegetable oil source.

In some embodiments, the lipid membrane comprises lipids and phospholipids derived from lecithin. When referring to “lipids and phospholipids derived from lecithin” it is to be understood as a lipid combination comprising at least two phospholipids, at least one of which is a PC, and at least one of which (which is or is not a PC) is characterized by one or more of the following features: (a) it has an unsaturated lipid tail; (b) it comprises a polar head group; (c) it comprises an acidic head group.

The plant lipids can be incorporated into the liposomes in their natural form (as they appear in nature) or they can be subjected to chemical modifications, such as, without being limited thereto, hydrogenation and oxidation.

In some embodiments, the liposome within the formulation disclosed herein comprise a lipid membrane composed of a combination of two or more liposome forming lipids, and the lipid membrane further comprises (i) at least one unsaturated lipid (that can be one of the liposome forming lipids or a non-liposome forming lipid) specifically unsaturated PC, (ii) PI; and (iii) sterol.

In accordance with some embodiments, the lipid membrane comprises at least a combination of (i) phospholipids that comprise PC, PI and PA; (ii) one or more sterols; and (iii) on or more saponins.

In some embodiments, the liposomes carry one or more (the same or different) agriculturally active agents or ingredients. In some embodiments, the one or more active agents are encapsulated within the intraliposomal internal aqueous core. In yet some other embodiments, the active agent is embedded in the lipid bilayer membrane.

Methods for loading of active agents within liposomes (either into the aqueous, intraliposomal core or within the lipid bilayer membrane) are well known in the art. The selection of a preferred loading technique, may depend, inter alia, on the type of active agent to be loaded, e.g. hydrophilic, hydrophobic, amphipathic, low molecular weight, macromolecule etc.

When referring to an agriculturally active agent, it is to be understood as any agent that provides a beneficial agricultural effect. The active agent may be a low molecular weight compound or a macromolecule, e.g. polymer.

The active agent may be classified according to its effect on the plant.

The active agent may be, but not limited to, pesticides, fertilizers, bio stimulants, and/or plant nutrients.

In some embodiments, the active agent is a pesticide. In the context of the present disclosure, when referring to a pesticide, it is to be understood as encompassing any substance used for destroying organisms harmful to cultivated plants.

In some embodiments, the pesticide is any member of the group consisting insecticides, herbicides, rodenticides, bacteriocides, fungicides and nematocides.

In some embodiments, the pesticide is a herbicide. Non limiting examples of herbicides include: Glufosinate, Propaquizafop, Metamitron, Metazachlor, Pendimethalin, Flufenacet, Diflufenican, Clomazone, Nicosulfuron, Mesotrione, Pinoxaden, Sulcotrione, Prosulfocarb, Sulfentrazone, Bifenox, Quinmerac, Triallate, Terbuthylazine, Atrazine, Oxyfluorfen, Diuron, Trifluralin, Chlorotoluron.

For example, and without being limited thereto, the herbicide can be used against weeds known to damage plants. For example, and without being limited thereto, the weeds can be any member of the following group of families: Gramineae, Umbelliferae, Papilionaceae, Cruciferae, Malvaceae, Eufhorbiaceae, Compositae, Chenopodiaceae, Fumariaceae, Charyophyllaceae, Primulaceae, Geraniaceae, Polygonaceae, Juncaceae, Cyperaceae, Aizoaceae, Asteraceae, Convolvulaceae, Cucurbitaceae, Euphorbiaceae, Polygonaceae, Portulaceae, Solanaceae, Rosaceae, Simaroubaceae, Lardizabalaceae, Liliaceae, Amaranthaceae, Vitaceae, Fabaceae, Primulaceae, Apocynaceae, Araliaceae, Caryophyllaceae, Asclepiadaceae, Celastraceae, Papaveraceae, Onagraceae, Ranunculaceae, Lamiaceae, Commelinaceae, Scrophulariaceae, Dipsacaceae, Boraginaceae, Equisetaceae, Geraniaceae, Rubiaceae, Cannabaceae, Hyperiacaceae, Balsaminaceae, Lobeliaceae, Caprifoliaceae, Nyctaginaceae, Oxalidaceae, Vitaceae, Urticaceae, Polypodiaceae, Anacardiaceae, Smilacaceae, Araceae, Campanulaceae, Typhaceae, Valerianaceae, Verbenaceae, Violaceae. For example, and without being limited thereto, the weeds can be any member of the group consisting of Lolium Rigidum, Amaramthus palmeri, Abutilon theopratsi, Sorghum halepense, Conyza Canadensis, Setaria verticillata, Capsella pastoris, and Cyperus rotundus. Additional weeds include, for example, Mimosapigra, salvinia, hyptis, senna, noogoora, burr, Jatropha gossypifolia, Parkinsonia aculeate, Chromolaena odorata, Cryptoslegia grandiflora, Anndropogon gayanus.

In some embodiment, the pesticide is a fungicide. Non limiting examples of fungicides include: azoxystrobin, mancozeb, prothioconazole, folpet, tebuconazole, difenoconazole, captan, bupirimate, fosetyl-A1.

In some embodiment, the pesticide is an insecticide. Non limiting examples of insecticides include Imidacloprid, Acetamiprid, Indoxacarb, Pymetrozine, Novaluron, Bifenthrin, Beta-Cyfluthrin, Spinosad, Acephate, Tau-Fluvalinate.

In some embodiments, the pesticide is a bacteriocide.

In some embodiments, the active agent is a plant nutrient. When referring to a plant nutrient it is to be understood as encompassing any substance that has a beneficial effect on the growth of the plant, substances necessary for plant growth and metabolism and completion of life cycle.

In some embodiments the plant nutrient is selected from the group consisting of macro-nutrients (N, P, K), secondary nutrients (S, Si, Ca, Mg) and micronutrients (Fe, B, Cl, Mo, Co, Cu, Zn, Ni, Al).

In some embodiments the plant nutrient is a plant hormone (phytohormone) or its metabolite or precursor, and plant growth regulators. In some embodiments, the phytohormone is selected from abscisic acid hormone, auxins, cytokinins, gibberellins, ethylene, brassinosteroids (polyhydroxysteroids), salicylic acid, jasmonates, plant peptide hormones, polyamines, nitric oxide, strigolactones, karrikins, and triacontanols.

The liposomes are carried by an agriculturally acceptable carrier.

In some embodiments, the “agriculturally acceptable carrier” is a carrier that is non-phytotoxic.

In some embodiments, the “agriculturally acceptable carrier” is a carrier that can be phytotoxic.

In some embodiments, the agriculturally acceptable carrier is selected from a polar organic solvent, water, water dispersible particulate matter (e.g. granules, capsules, beads, pellets, tablets, etc.).

In some embodiments, the agriculturally acceptable carrier is inert, i.e. while it may facilitate in the delivery of the liposomes to the plant (e.g. in penetration), it does not abrogate the integrity of the liposomes and/or the activity of any active agent carried by the formulation, and preferably within the liposomes.

The selection of the carrier may depend on the manner of bringing the formulation into contact with the plant, as further discussed below.

The present disclosure also provides a method for treating in the field of agriculture. In some embodiments, the treatment can be of a plant or plant part.

In some other embodiments, the treatment can be via application of the formulation to the soil, or to a plant growth medium (e.g. hydroponic growing medium).

In some embodiments, the method is for treating a plant and the method comprises applying to the surface of the plant the formulation disclosed herein.

In the context of the present disclosure “treating” denotes an effect on the plant that can be for reducing, inhibiting or eliminating a plant pathological condition, be it one caused by a pathogen or by an environmental condition (physiological factors); or for preventing from the pathological condition from developing. Thus, in the context of the present disclosure, treatment encompasses treatment for curing from a pathological condition, as well as protective treatment.

In some embodiments, the treatment is for pest control.

In some embodiments, the treatment is for improving crop.

In some embodiments, the treatment is for imparting the plant with a desired trait. In the context of the present disclosure, a plant trait can include, without being limited thereto, abiotic or biotic stress tolerance, drought tolerance, high harvest yield, high biomass and/or vigor, high seed yield/quality, increased crop/flower per plant, growth rate, fruit quality (fruits color break, firmness, shine, etc) etc.

The liposomal formulation disclosed herein is to be applied to the plant by direct contact with the plant or part thereof. Direct contact requires that intact liposomes within the formulation are in contacted with the plant or plant part.

In the context of the present disclosure, a “plant part” denotes any one or combination of the meristems, leaves, root, stem, shoot, flower, fruit, tuber, seed, onion, petriole, bud, tendril, trunk, bulb, rhizome and stolon.

It has been found, as also exhibited in the following non-limiting examples, that when applied onto the plant's leaves, the formulation effectively penetrates and is distributed from the leaves to the plant parts, including the apical shoot. Thus, in some embodiments, the formulation is applied onto at least a portion of the plant's foliage.

Thus, in the context of the present disclosure, the liposomes effectively penetrate into the plant, and are distributed throughout portions of the plant.

Specifically, in the context of the present disclosure, when referring to “penetration” it is understood to encompass the translocation of intact liposomes from the external surface of the plant part that has been brought into contact with the formulation, into the plant, via the plant cuticles, epidermis or hypodermis.

Penetration into a plant part can be determined by measuring the amount of a detectable liposome component, e.g. a marked membrane lipid or other membrane component or a marked encapsulated agent, after the plant part has been thoroughly rinsed. The mark can be by the use of a fluorescent dye.

Further, in the context of the present disclosure, when referring to “distribution” it is to be understood to encompass the translocation of at least the active agent from the plant part onto which the formulation has been applied, to at least one other plant part.

In some embodiments, distribution is of intact liposomes.

Distribution of the active agent and/or intact liposomes carrying the active agent can be determined, for example, by thoroughly rinsing or entirely removing the plant part onto which the formulation has been applied and measuring the amount of a detectable liposome component, e.g. a marked membrane lipid or other membrane component or a marked encapsulated agent. Also in this case, the mark can be by the use of a fluorescent dye. Distribution can be to any plant part, such as apical shoot, leaves, stem and roots.

In some embodiment, distribution is at least to the apical shoot.

Delivery of the formulation to the plant's part can be by any one or combination of spraying the plant, smearing the formulation onto the plant part, submerging the plant part within the formulation, fumigation, applying ultrasonic droplets, dusting with the formulation.

In some embodiments, the formulation is applied by spraying. To this end, and in accordance with some embodiments, the formulation is in a form of an aqueous formulation in which the liposomes are suspended or dispersed. The aqueous formulation may also contain, suspended therein, particulate matter (e.g. bead, capsules, etc.) carrying the liposomes.

In some embodiments, the liposomal formulation is applied onto the plant part by smearing.

In some embodiments, the liposomal formulation is applied onto the plant part by submerging the plant part, e.g. leaves, into the liquid formulation.

In some other embodiments, the formulation is applied onto the plant's seeds. According to this embodiment, and without being limited thereto, the seeds can be sprayed with the formulation and/or be submerged within or drenched by the formulation.

In some embodiments, the formulation is applied to the plant leaves. This may be achieved by any of the above listed delivery techniques, i.e. smearing, spreading, spraying, immersing, fumigating, applying US droplets, dusting. In some embodiments, the leaves are sprayed with the formulation.

In some other embodiments, the formulation is applied onto the plant's roots. According to this embodiment and without being limited thereto, direct contact of the roots with the formulation can be achieved by the use of hydroponic systems with the formulation being dispersed or suspended in the plant reservoir within the hydroponic tank or tray.

In some embodiments, the liposomes are delivered to the plant through irrigation.

The formulation disclosed herein is applied onto the plant or plant part in an amount, and at a schedule that is effective to treat the plant. The amount and schedule can be easily determined by those versed in the art and will depend, inter alia, on the type of the plant, the pathology, whether it is protective or curative treatment, the environmental conditions etc.

Also disclosed herein is the use of the formulation for agricultural applications, e.g. in accordance with the method disclosed herein.

In addition, disclosed herein is a kit comprising (a) an agriculturally acceptable carrier; (b) liposomes or liposome forming lipids as defined herein; and (c) instructions for use of the carrier and liposomes for treating a plant.

In some embodiments, the liposomes within the kit are in dry form, e.g. lyophilized and the instructions comprise, steps for rehydrating the liposomes together with the carrier.

In some embodiments, the kit comprises separately, the lipid membrane components, i.e. the liposome forming lipids and other lipid components as defined herein with the active agent being within the agriculturally acceptable carrier, such that when mixed together, liposomes encapsulating the active agent are formed.

As used herein, the forms “a”, “an” and “the” include singular as well as plural references unless the context clearly dictates otherwise. For example, the term “a liposome” includes one or more liposomes.

Further, as used herein, the term “comprising” is intended to mean that the formulation includes the recited liposome and carrier but not excluding other elements, such as a surfactant or other components that may be part of the liposome or part of the carrier. The term “consisting essentially of” is used to define formulations which include the recited elements but exclude other elements that may have an essential significance on the formulation. “Consisting of” shall thus mean excluding more than trace elements of other elements. Embodiments defined by each of these transition terms are within the scope of this invention.

Further, all numerical values, e.g. when referring the amounts or ranges of the elements constituting the formulation are approximations which are varied (+) or (−) by up to 20%, at times by up to 10% of from the stated values. It is to be understood, even if not always explicitly stated that all numerical designations are preceded by the term “about”.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### SOME NON LIMITING EXAMPLES

### Materials and Methods

Materials:

L-α-Phosphatidylcholine, hydrogenated from soy bean (HSPC) was obtained from Avanti Lipids, Inc.

Soy lecithin was obtained from various commercial suppliers

Cholesterol was obtained from Sigma Aldrich.

1,2-Distearoyl-sn-glycero-3-phosphoethanolamine-N-[amino(polyethylene glycol)-2000] (PEG-DSPE 2000) as an ammonium salt was obtained from Lipoid.

Calcium Acetate (CaAc), Fluorescein, EuCl₃, Mannitol, MES, Cellulase R10, 0.66-0.8% Macerozyme R10, and 0.1% BSA were obtained from Sigma Aldrich.

Sequesterene 138 (iron chelate microelement nutrient) was obtained from 15 Syngenta.

MgSO₄ was obtained from Merck.

All other reagents and solvents were obtained from known vendors.

Methods:

Preparation of Liposomes

Extraction of Lipids from Soy Lecithin for Liposomes B

Lipids are extracted from soy lecithin by dissolving the lecithin in EtOH and heating to 60° C. for approximately an hour. The upper liquid phase is collected and the EtOH evaporated. The lipid mixture powder obtained comprises phosphatidylcholine (PC)—35-50%; phosphatidylinositol (PI)—10-20%; phosphatidic acid (PA)—3-6%; phytosterols and saponins—25-30%. The chain types in the mixture comprise 16:0-20-25%; 18:0-10-15%; 18:1-15-22%; 18:2-35-40%; 18:3-10-15%.

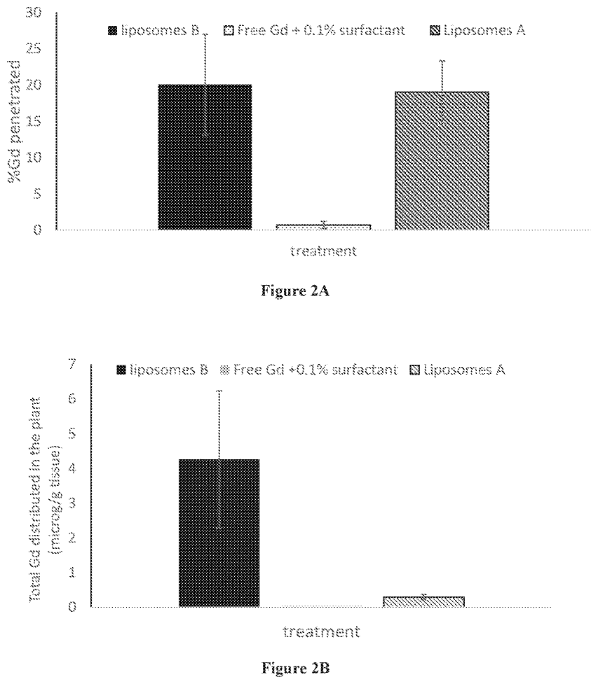

Lipids extracted from lecithin by this method were used for preparation of Liposomes B, for the experiments presented in FIGS. 2A-2B, FIG. 6C, FIG. 7C, FIG. 8B, FIG. 10, FIG. 11A-11B.

Liposome Production by Ethanol Injection with Passive Loading

An organic phase is prepared by dissolving a selected lipid formulation in a water-miscible organic solvent such as EtOH (10% volume or less from the final volume of a combined organic and inorganic emulsion) at a temperature above the gel-to-liquid crystalline phase transition temperature (Tm) of the dominant lipid (e.g., 65° C. for HSPC) to obtain a concentration of 50 mM.
The lipid mixture for Liposome A included a single fully saturated phosphatidyl choline (PC) lipid. Unless otherwise stated, Liposome A comprises, as its basic composition the following Table 1:

TABLE 1
Liposome A composition

| Component | Mol % | % w/w |
|---|---|---|
| HSPC (with carbon chain type 88.6% 18:0, | 60 | 70 |
| 11.4% 16:0) | | |
| Cholesterol | 38 | 21 |
| PEG-DSPE | 2 | 9 |

The lipid mixture for Liposome B included lipids extracted from soy lecithin as described above; soy phytosterol comprising β sitosterol—˜35%, stigmasterol ˜25%, ergosterol ˜39%; and PEG-The breakdown of the sterol component depended on the ratio of lecithin and added phytosterol. Unless otherwise stated, Liposome B were prepared from 39.5 mM lipids extracted from soy lecithin as described above, 9.5 mM soy phytosterol and 1 mM PEG-DSPE.

An aqueous phase is prepared separately by dissolving the compound (agent) to be encapsulated in water at 65° C.

The organic and aqueous phases are then merged by injecting the organic phase into the inorganic phase, preferably in a rapid and consistent motion, to thereby obtain a cloudy solution (an emulsion), which is vortexed for a few seconds. Vesicles encapsulating the compound of interest are spontaneously formed, and are thereafter downsized by stepwise extrusion through 400, 100, 80 nm membranes (5 repetitions for each membrane). The extruded solution is then subjected to dialysis (at e.g., 12-14 kD cutoff) at room temperature.

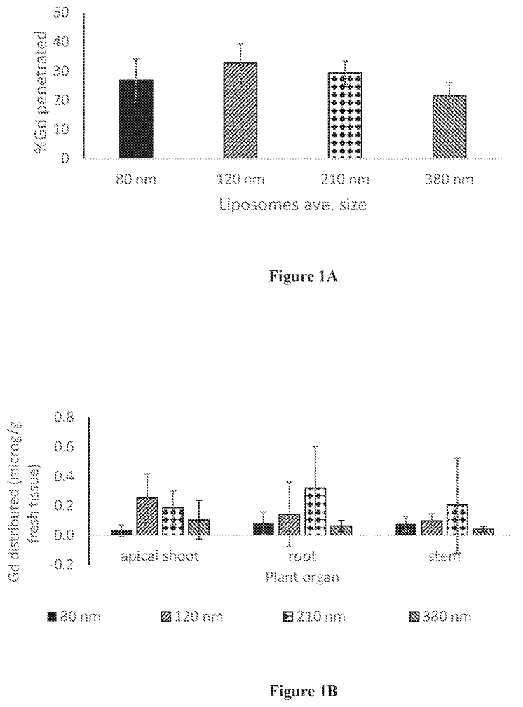

This method was used for preparation of Liposomes A, B for the experiments presented in FIGS. 1-7 and 9-14.

### Production of Gd-Encapsulating Liposomes a, B

Liposomes A, B were prepared according to the passive loading procedure described hereinabove, using an aqueous phase prepared by dissolving Diethylenetriaminepentaacetic acid gadolinium(III) dihydrogen salt hydrate in aqua solution, to achieve a final concentration of 100 Mm Gd. The obtained concentration of Gd in the liposomes was approximately 2 mM.

Production of EuCl₃-encapsulating Liposomes A

Liposomes A were prepared according to the passive loading procedure described hereinabove, using an aqueous phase prepared by dissolving EuCl₃ in a pre-prepared 5% DEX solution, to achieve a final concentration of 50 mg/ml. The obtained concentration of EuCl₃ in the liposomes was approximately 2 mg/ml

Production of MgSO₄-encapsulating Liposomes A

Liposomes A were prepared according to the passive loading procedure described hereinabove, using an aqueous phase prepared by dissolving 20% w/w MgSO4.7H2O in a pre-prepared 5% DEX solution, to achieve a final MgSO₄ concentration of about 2% wt. in the liposomes, which is in line with the commonly-used amounts of this fertilizer in traditional plant fertilizing.

### Production of Chelated and Non-Chelated Iron-Encapsulating Lioposomes A

Liposomes A were prepared according to the passive loading procedure described hereinabove, using an aqueous phase prepared by dissolving 16-17% w/w Sequestrene™ 138 (chelated iron) in a pre-prepared 5% DEX solution, to achieve a final Sequesterene-derived iron concentration of about 0.1% wt. in the liposomes, which is in line with the commonly-used amounts of this fertilizer in traditional plant fertilizing.

Liposomes encapsulating non-chelated iron were prepared in a similar manner. The aqueous phase was prepared by mixing a 10 grams/liter solution of AAS-grade Fe-standard in a pre-prepared 5% DEX solution, to achieve a final Iron concentration of about 0.1% wt. in the liposomes.

### Production of Fluorescein-Encapsulating Liposomes A

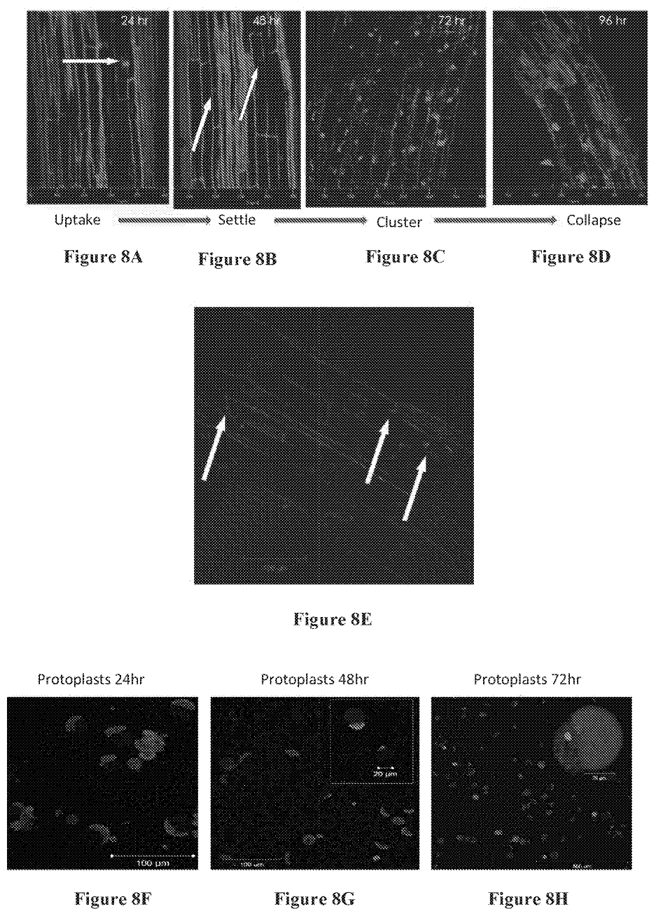

Fluorescein-encapsulating Liposomes A for the experiment presented in FIGS. 9A-9C were prepared by active loading, which is a method that is typically used for encapsulating weak acids or weak bases, or amphiphatic (amphiphilic) compounds that have a charged and an un-charged form that can be dependent on pH or other conditions of 5 the media.

Liposomes encapsulating CaAc-encapsulating are first prepared by ethanol injection and passive loading, as described above, wherein the concentration of the lipid formation was 50 mM and the aqueous (inorganic) phase was prepared separately by dissolving CaAc in water at 65° C., at a concentration of 100 mM. Following successive encapsulation of CaAc and dialysis (performed in order to dismiss excess non-encapsulated CaAc), CaAc-containing liposomes are introduced into a 2 mg/ml 5 Fluorescein-containing (free acid, from Sigma) 5% D+-Glucose (Dextrose, DEX) solution in a 1:2 ratio (such that liposomes are diluted 1:2 within the solution, and the final concentration of Fluorescein post-mixing is 1 mg/ml), at 55° C., and the obtained mixture is subjected to constant magnetic stirring for 60 minutes. The Fluorescent compound is then mobilized by its concentration gradient through the 10 temperature-disturbed liposomal membranes, where conjugation with Ca occurs causing the newly formed salt to precipitate inside the particle. The obtained liposome solution is thereafter left to cool to room temperature and further dialysis is performed in order to remove non-precipitated/non-encapsulated dye. The obtained concentration of fluorescein in the liposomes was approximately 0.6 mg/ml

### Determining Liposome Characteristics

### Size Measurement

Liposomes were measured using Dynamic Light Scattering (DLS) instrument (http://www.malvern.com/en/products/technology/dynamic-light-scattering/) for of particle size characterization and size distribution. Samples were diluted 1:100 with the buffer in which the liposomes were prepared.

### Zeta Potential

Zeta potential is a measure of the magnitude of the electrostatic or charge repulsion/attraction between particles, and is one of the fundamental parameters known to affect stability. Zeta potential for liposomes solution was measured using DLS instrument. Samples were diluted in ratio of 1:100 with the buffer which the liposomes were prepared in.

### Determination of Encapsulated Materials

### Pyranine (HPTS)

To quantify pyranine entrapment, liposomes encapsulating pyranine were decomposed with 0.1% triton and pyranine was determined using Tekan, Multimode Microplate Reader with fluorescence in excitation wavelength 413 nm and emission wavelength 510 nm.

### Fluorescein

To quantify fluorescein entrapment, liposomes encapsulating fluorescein were decomposed with 0.1% triton and fluorescein was determined using Tekan, Multimode microplate reader with absorbance in wavelength of 525 nm.

### Metals (Gd, Eu, Mg, Fe)

To quantify metal entrapment, liposomes encapsulating metals were dissolved in 1% HNO3 at a ratio of 1:100, vortexed and filtered through a 0.45 μm syringe filter. Metals were determined using Inductively Coupled Plasma Optical Emission Spectroscopy (ICP OES). Wavelengths used were 342 nm for Gd, 397 nm for Eu, 280 nm for Mg and 259 nm for Fe.

### Glufosinate

To quantify glufosinate entrapment liposomes were decomposed using Bligh and Dayer method, Glufosinate being dissolved in the upper phase. Glufosinate was determined using High Performance Liquid Chromatography with Diode-Array Detection (HPLC-DAD) according to the method described in Changa et. al, Journal of the Chinese Chemical Society, 2005, 52, 785-792. Briefly, 9-fluorenylmethyl chloroformate (FMOC-Cl) was used for pre-column derivatization of the non-absorbing glufosinate. The samples were separated by HPLC-DAD at 12 min with 25 mMborate buffer at pH 9, followed by determination with a UV detector at 260 nm.

### Application of Liposomes to Plants

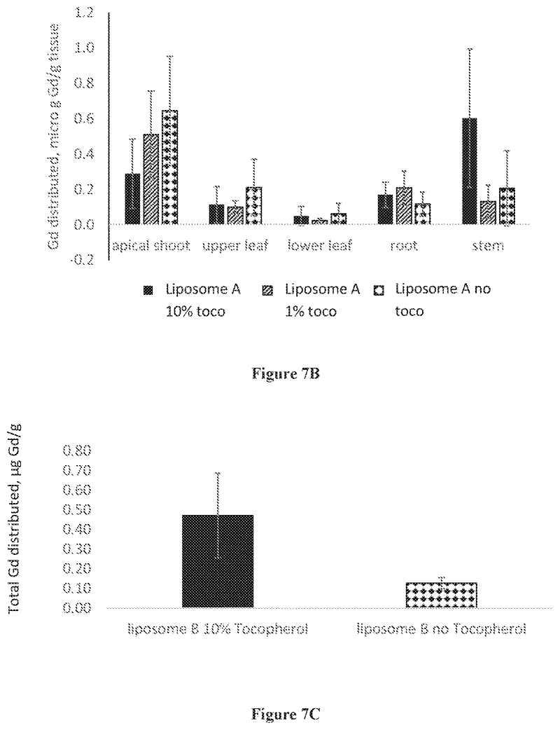

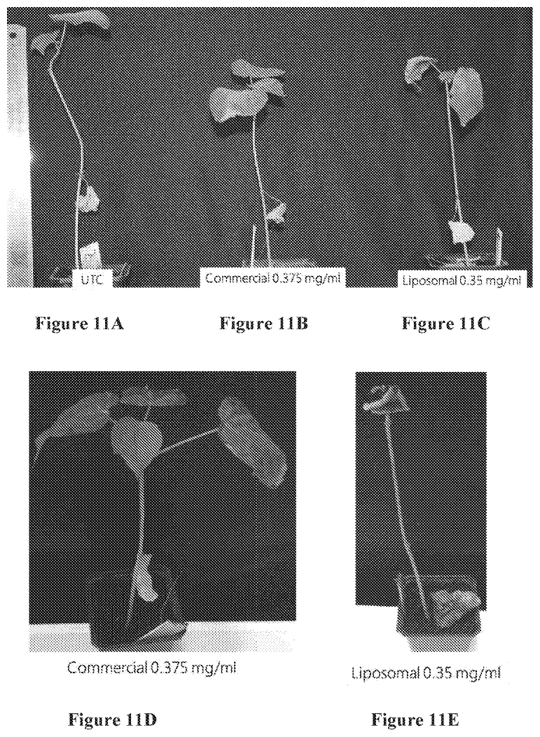

Liposomes were applied to plants using three different application methods
Leaflet submerging (foliar absorption): Submerging one leaflet in an Eppendorf vial containing the liposome solution for 72-96 h. The plant remains planted in soil or submerged in a hydroponic solution throughout the experiment. This method was used in the experiments described in FIGS. 8, 9.
Smearing: Liposomal solution is gently dripped on a single leaf, and then spread by the flat side of a pipettor tip. The plant remains planted in soil or submerged in a hydroponic solution throughout the experiment. This method was used in the experiments described in FIGS. 1-7 and FIGS. 10, 12-14.
Spraying: Approximately 1 ml Liposomal solution is applied on all plant foliage (above soil surface) using an aerosol sprayer, until dripping. This method was used in the experiments described in FIG. 11.

### Microscopy

At the determined time following liposomal application, plants were sliced into different organs, washed and counterstained using propidium iodide solution 75 mM. Samples were examined in the Confocal Zeiss LSM 510 META.

### Quantification of Bio-Distribution

### Quantification of Penetration and Distribution in Tomato Plants

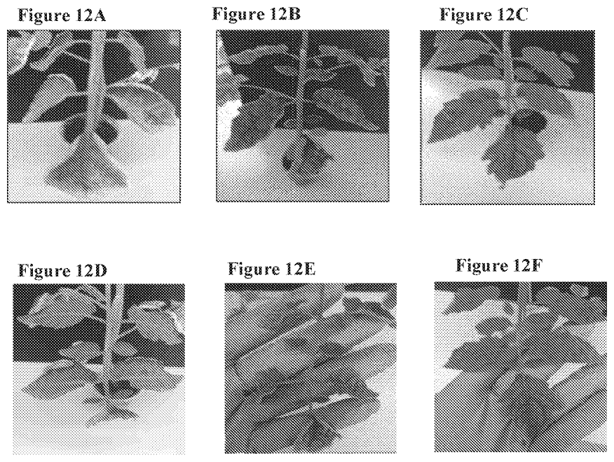

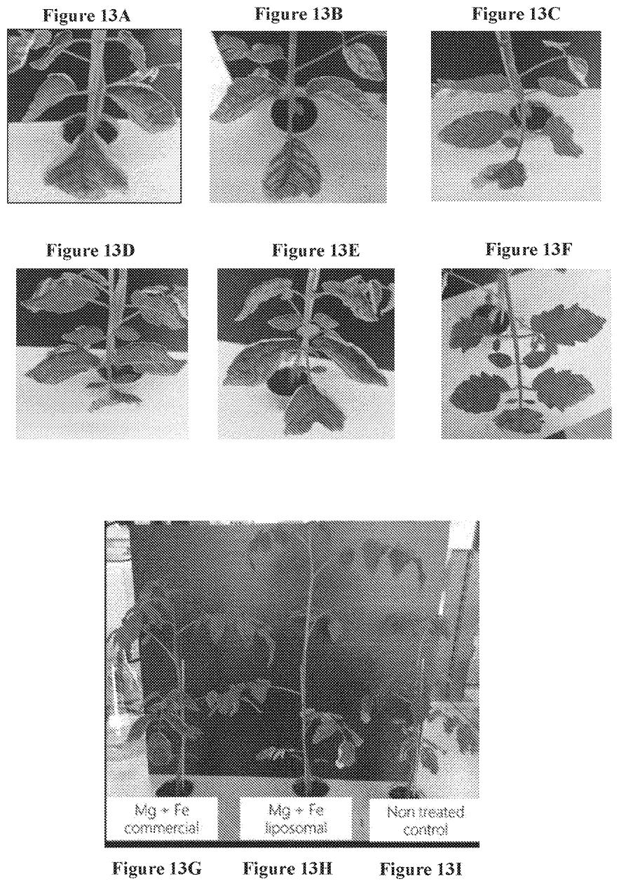

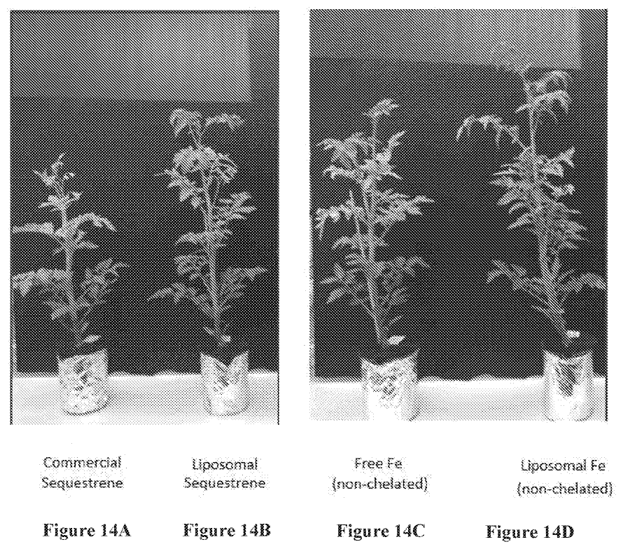

Cherry tomato (Shiren variety) seeds of uniform genetics were germinated and grown in a designated nursery in soil (experiments presented in FIGS. 12, 13) or in a Hoagland hydroponic solution (experiment presented in FIG. 14) for 3 weeks, until physiological age of 7 leaves.

Gd-encapsulating Liposomes A, B (concentration of 2 mg/ml) were prepared as described above and 0.1 ml was applied to a single leaf by smearing as described above.

72 hours after application, the leaf on which the formulation was applied was thoroughly rinsed for 45 seconds, under running water. Plants were thereafter cut and divided to leaflet samples, petiole samples, stem sample and root sample, samples were dehydrated in oven (2 hours at 105° C.) and the dry weight recorded. The dried samples were placed in ceramic bowls and fully digested by cremation for 3-5 hours at 550° C. Ash residues were dissolved in 1% nitric-acid and collected to tubes, at a final volume of 10 ml for each sample. Samples were filtered through 0.45 μm syringe filter and analyzed using Inductively Coupled Plasma Optical Emission Spectroscopy (ICP OES) with a wavelength of 342 nm.

Penetration was calculated as the % of Gd applied that was found in the entire plant, including the leaf on which the formulation was applied.

Total distribution is the amount of Gd (microgram Gd/gram fresh tissue) found in the entire plant excluding the leaf on which the formulation was applied. Distribution to different organs is the amount of Gd (microgram Gd/gram fresh tissue) found in each organ separately.

### Quantification of Lateral Translocation in Mature Vine

The objective was to determine movement of liposome in woody plants (mature vine in vineyard), as well as to evaluate the movements from the younger to older plant parts (from top of the branch toward the base).

Eu-encapsulating Liposomes A (concentration of 50 mg/ml) were prepared as described above.

The liposomes were applied to the youngest leaf of a mature vine branch by the leaflet submerge method, as described above.

After application (72 hours), sample vine leaves were collected at distances of 5 cm from the application point downward toward the branch base, a distance of 60 cm in total. Samples were dehydrated in oven (2 hours at 105° C.) and the dry weight recorded. The dried samples were placed in ceramic bowls and fully digested by cremation for 3-5 hours at 550° C. Ash residues were dissolved in 1% nitric-acid and collected to tubes, at a final volume of 10 ml for each sample. Samples were filtered through 0.45 μm syringe filter and analyzed using Inductively Coupled Plasma Optical Emission Spectroscopy (ICP OES) with a wavelength of 280 nm.

### Assessment of Deficiency Correction

Liposomes A encapsulating MgSO₄, Fe and Sequestrene™ were prepared as described above.

Hoagland solution formulation was prepared according to Epstein, E. Mineral Nutrition of Plants: Principles and Perspectives. John Wiley & Sons, Inc. 1972, pp. 412, with some modification, using the ingredients listed in Table 2 below.

TABLE 2
Hoagland solution formulation

| | | | |
|---|---|---|---|
| | 0.5 Hoagland | Stock | Stock |
| | solution | x10 | x1000 |
| Component | (optimum) [mg/l] | [g/l] | [g/l] |

Macroelements

| | | |
|---|---|---|
| Ca(NO₃)₂*4H₂0 | 720 | 7.2 |
| KNO₃ | 460.98 | 4.61 |
| MgSO₄*7H₂O | 493 | 4.93 |
| KH₂PO₄ | 272 | 2.72 |
| Iron (Sprint 138 iron chelate) | 100 | 1.0 |

Microelements

| | | | |
|---|---|---|---|
| H₃BO₃ | 2.86 | 0.0209 | 2.09 |
| MnCl₂*H₂O | 1.8 | 0.018 | 1.8 |
| ZnSO₄*7H₂O | 0.22 | 0.0022 | 0.22 |
| CuSO₄ | 0.08 | 0.0008 | 0.08 |
| NaMoO₄*2H₂O | 0.017 | 0.00017 | 0.017 |

Cherry tomato (Sheran variety) seeds of uniform genetics were germinated and grown in designated nursery for about 3 weeks (e.g., until generation of 4th leaf), stripped from soil and washed thoroughly with DDW. Each plant's exposed root system was immersed in 250 ml of full 0.5 Hoagland solution, as described above, and air-pumped constantly for 7 days under steady ambient temperature, humidity and CO2 levels (data recorded by Rotronic CL11 sensor). After adaptation of the plants to full Hoagland media, plants were taken out of the beakers (excluding Group 3), washed thoroughly with DDW and transferred to a premixed microelement-deficient Hoagland solution (Mg-deficient for the experiments presented in FIGS. 12, both Mg and Fe-deficient for the experiment presented in FIG. 13, and Fe-deficient for the experiment presented in FIG. 14).

Plants were visually examined daily for signs of nutrient deficiency.

Once deficiency was identified, commercial and liposomal formulations were applied by smearing on a single leaf:
Experiments described in FIG. 12: Either commercial unformulated MgSO₄ or Liposome A containing MgSO₄ were applied to the topmost leaf
Experiments described in FIG. 13: Either a mixture of commercial unformulated MgSO₄ and free Fe, or a mixture of Liposomes A containing MgSO₄ and free Fe (encapsulated separately) were applied to the topmost leaf
Experiments described in FIG. 14: Free Fe, Sequestrene™, Liposomes A containing free Fe or Liposomes A containing Sequestrene™ were applied to the lowest leaf

Ten days after application, the plants were examined for signs of deficiency-interveinal chlorosis and epinasty of older leaves, and overall growth and appearance were compared with untreated controls.

Assessment of Glufosinate Activity

Liposomes B encapsulating glufosinate were prepared as described above.

Cotton plant seeds of uniform genetics were germinated and grown in designated nursery for 6 weeks (2-3 leaves).

Either commercial formulation of glufosinate or glufosinate-encapsulating Liposome B were applied by spraying as described above.

Glufosinate activity was assessed phenotypically (signs of chlorosis and wilting, necrosis, plant death) on days 22 and 35 after treatment.

### Example 1—Effect of Liposome Composition on Penetration and Distribution of Gd in Tomato Plant

In the following examples, Gd was used either in free form or encapsulated in Liposome A or Liposome B, with variations in liposome composition, as specifically indicated.

For the purpose of quantifying penetration and distribution of Gd into various organs of the tomato plant, liposome formulation was smeared on a single leaf of 4-8 weeks old tomato plants, at a physiological age of 4-7 true leaves. Unless otherwise stated, 72 hours after application, the plants were dissected and cremated. Concentration of Gd in different plant organs was detected by ICP-OES. The plant organs examined were: apical shoot, leaf above loading point, leaf below loading point, roots and stem. Overall, the entire plant was cremated.

### Effect of Liposome Size

FIGS. 1A-1B present penetration and distribution, respectively, of Liposome A of different sizes encapsulating Gd. It is shown that the best penetration and distribution was obtained with liposome sizes between an average size of 210 nm (50% of liposomes in the size range of 170-250 nm) to an average size of 120 nm (50% of liposomes in the size range of 90-150 nm).

Comparison Between Effect of Free Gd in 0.1% Triton Vs. Liposomal Gd on Penetration and Distribution

FIG. 2A shows that Gd penetration was enhanced when encapsulated in Liposome A or Liposome B as compared to its free form in water containing 0.1% Triton v/v.

However, FIG. 2B shows that Gd encapsulated in Liposome B showed a significantly better distribution over Gd encapsulation in Liposome A, or in free form. This suggests that Liposome B enables more of the cargo to move out of the leaf on which it is applied, to other plant organs.

### Effect of Cholesterol in Lipid Membrane

For the purpose of determining the effect of cholesterol concentration on penetration and distribution, the following liposome compositions were compared:

TABLE 1
Cholesterol in variants of Liposome A compositions

| Component | Mol % (% w/w) | | | | | | | |
|---|---|---|---|---|---|---|---|---|
| Cholesterol | 0% | | 19% | (9% w/w) | 25% | (13% w/w) | 49% | (30% w/w) |
| HSPC | 98% | (93% w/w) | 79% | (83% w/w) | 73% | (77% w/w) | 49% | (62% w/w) |
| PEG-DSPE | 2% | (7.5% w/w) | 2% | (8% w/w) | 2% | (9% w/w) | 2% | (8% w/w) |

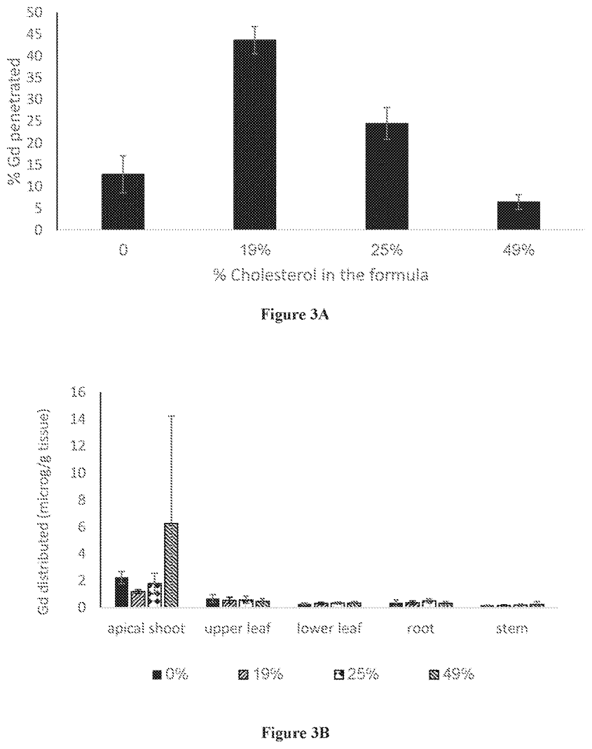

FIGS. 3A and 3B show that while for penetration 19% cholesterol in the liposomal formulation is more effective, for distribution of Liposome A it is preferred to have higher cholesterol concentration, even up to 50%.

### Effect of PEG-DSPE

For the purpose of determining the effect of PEG-DSPE on penetration and distribution of Liposomes A, PEG-DSPE in the indicated amounts were added, as indicated in Table 3 below:

TABLE 3
PEG-DSPEC in Liposome A composition

| | |
|---|---|
| Component | Mol % (% w/w) |

| | | | |
|---|---|---|---|
| PEG-DSPE | 0% | 2% | (9% w/w) |

| | | | | |
|---|---|---|---|---|
| HSPC | 62% | (77% w/w) | 60% | (70% w/w) |
| Cholesterol | 38% | (23% w/w) | 38% | (21% w/w) |

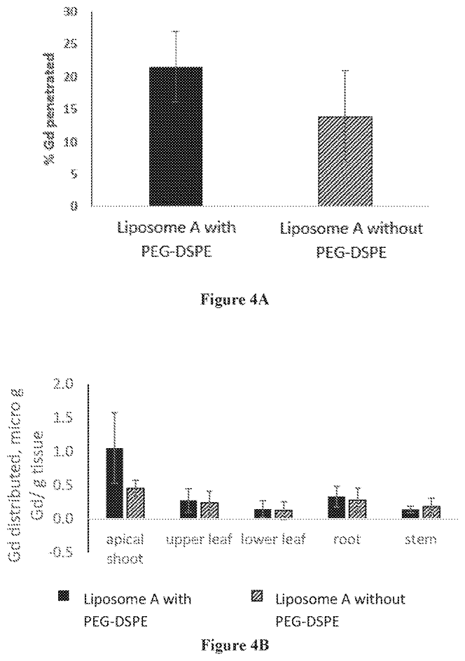

FIGS. 4A and 4B show that the presence of PEG-DSPE in the liposomal formulation may be advantageous (although not mandatory), particularly for distribution to the apical shoot.

### Effect of Lipid Chain Length

For the purpose of determining the effect of chain length on penetration and distribution, phospholipids with different chain lengths were used to prepare Liposome A (each variant of Liposome A comprised a single type of chain):
HSPC (18:0) (“lipo HSPC”)
DPPC (16:0) (“lipo DPPC”)
DMPC (14:0) (“lipo DMPC”)

The lipid mixture consisted of one of the phospholipids listed above (60 mol %), cholesterol (38 mol %) and PEG-DSPE (2 mol %).

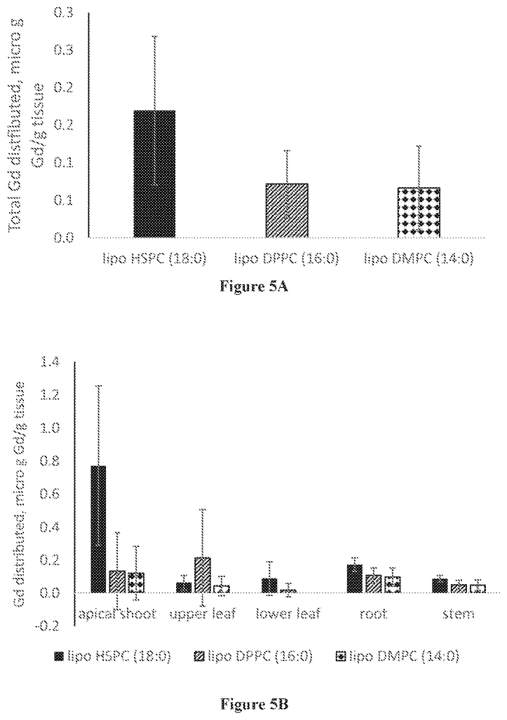

FIG. 5A-5B show that chains of 18 carbons provide better distribution as compared to liposomes composed of a shorter chain, with the main effect found in the translocation to the apical shoot.

### Effect of Positively Charged Lipid, DOTAP

In the following, the effect of N-[1-(2,3-Dioleoyloxy)propyl]-N,N,N-trimethylammonium) (DOTAP) incorporated into liposome A or Liposome B, on the distribution was determined. The liposomal composition is presented in Tables 4A-4B:

TABLE 4A
DOTAP in Liposome A composition

| Component | Mol % (% w/w) | | | |
|---|---|---|---|---|
| DOTAP | 10% | (10% w/w) | 0% | |
| HSPC | 50% | (60% w/w) | 60% | (70% w/w) |
| Cholesterol | 38% | (21% w/w) | 38% | (21% w/w) |
| PEG-DSPE | 2% | (8% w/w) | 2% | (8% w/w) |

TABLE 4B
DOTAP in Liposome B composition

| Component | Mol % (% w/w) | | | |
|---|---|---|---|---|
| DOTAP | 0% | | 15% | (19% w/w) |
| Phospholipids | 50% | (62% w/w) | 36% | (43% w/w) |
| Phytosterols & saponins | 48% | (31% w/w) | 46% | (28% w/w) |
| PEG-DSPE | 2% | (7% w/w) | 2% | (10% w/w) |

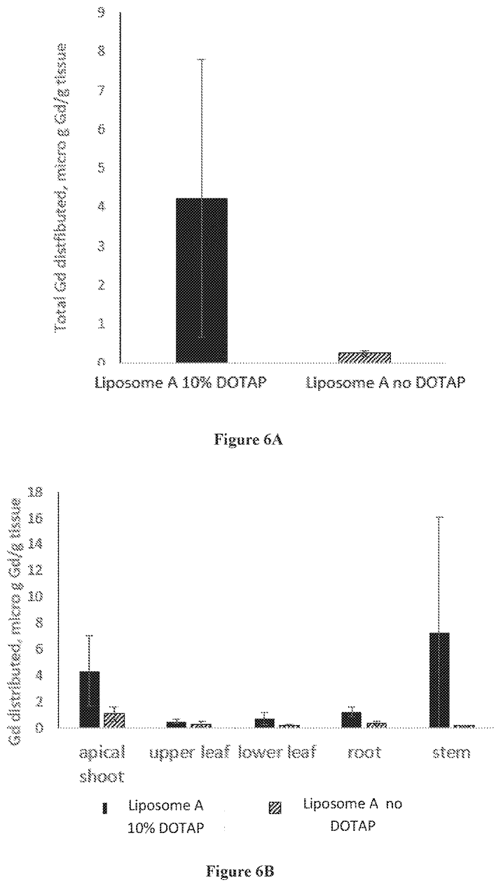

FIGS. 6A-6B show that incorporating 10% DOTAP in Liposome A increased distribution, especially to the apical shoot and the stem.

When incorporated into Liposome B, an even stronger positive effect was observed, where distribution even to the root was significantly high, as shown in FIG. 6C.

Notably, Liposome B, being made of soy lecithin has a negative charge. The addition of 15% DOTAP to Liposome B has changed the zeta potential positively by 10 my, but the zeta of the liposomes was nevertheless negative—−20 my.

### Effect of α-Tocopherol

In the following, the effect of α-tocopherol incorporated into Liposome A or Liposome B, on the distribution was determined. The liposomal compositions are presented in Tables 5A-5B:

TABLE 5A
α-tocopherol in Liposome A composition

| Component | Mol % (% w/w) | | | | | |
|---|---|---|---|---|---|---|
| α-tocopherol | 10% | (7% w/w) | 1% | (1.5% w/w) | 0% | |
| HSPC | 50% | (62% w/w) | 59% | (69% w/w) | 60% | (70% w/w) |
| Cholesterol | 38% | (22% w/w) | 38% | (21% w/w) | 38% | (21% w/w) |
| PEG-DSPE | 2% | (9% w/w) | 2% | (8.5% w/w) | 2% | (8% w/w) |

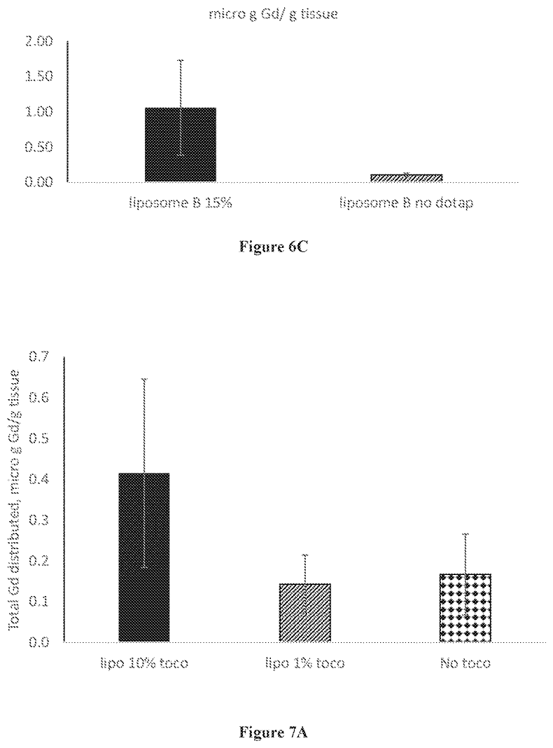

The effect of α-tocopherol is shown in FIGS. 7A-7B, where an increase in distribution was greater with 10% α-tocopherol as compared to 1% α-tocopherol or no α-tocopherol in the formulation. The beneficial effect was even more pronounced from the distribution of 10% α-tocopherol from the leaf onto which the formulation was applied to both the apical shoot and stem.

TABLE 5B
α-tocopherol in Liposome B composition

| Component | Mol % (% w/w) | | | |
|---|---|---|---|---|
| α-tocopherol | 10% | (6% w/w) | 0% | |
| Phospholipids | 45% | (57% w/w) | 50% | (62% w/w) |
| Sterols & similar | 43% | (30% w/w) | 48% | (31% w/w) |
| PEG-DSPE | 2% | (7% w/w) | 2% | (7% w/w) |

As shown in FIG. 7C, a similar beneficial effect on penetration was observed when 10% α-tocopherol was incorporated into Liposome B.

### Example 2—Translocation Kinetics

### Translocation of Fluorescein

In the following examples, Liposome A encapsulating Fluorescein (fluorescent marker) was applied using leaflet-submerging method on tomato plants. Liposome presence in the roots was detected using confocal microscopy 24-96 hours after application.

FIG. 8A shows that 24 hours post application liposomes (appearing as liposome aggregates) are observed in the a few cells of the roots (several of the aggregates marked by arrows). Yet, 72 hours after application these aggregates were already present in most of the observed root cells; and 96 hours after application the liposomes seemed to have collapsed thereby releasing their cargo, this being evident by the coloring of the entire cell.

Similar results were obtained when pyranine was encapsulated within Liposome B (phospholipids 51%, phytosterol 47%, PEG-DSPE 2%, where presence of the fluorescent marker in the roots, 72 hours post application was observed, as shown in FIG. 8B.

In a similar experiment, the presence of Fluorescein encapsulated in Liposome A in adjacent leaves was observed after 24 hr, 48 hr and 72 hr, as shown in FIG. 8C. Protoplasts from the leaf adjacent to the leaf to which the liposomes were applied are presented. 24 and 48 hrs after application, liposome aggregates are present in the cells. 72 hrs after application the liposomes collapse and the protoplasts glow in brightly from the release of the fluorescein.

### Translocation of EuCl₃

The following experiment demonstrated the translocation of Europium in mature woody plants. To this end, a single vine leaf was submerged in a solution containing Europium encapsulated in Liposomes A.

As shown in FIG. 9, Eu was detected 60 cm from the application point, supporting the finding that liposomes are effective in distributing encapsulated cargo Throughout the Plant Parts.

### Example 3—Herbicide Delivery

### Translocation of Herbicidal Glufosinate

Three plants of Eleusine indica were grown in the same pot. Fourteen days after germination, each plant was subjected to a different herbicidal treatments by smearing a single leaf of each plant. Specifically, the following groups were examined:

Left Plant: Plant treated with a commercial Glufosinate herbicide at the recommended dose (6 mg gluofosinate/ml);

Center Plant: Plant treated with Liposome B encapsulating Glufosinate at 65% of its recommended dose (3.9 mg glufosinate/ml).

Right Plant: non-treated plant.

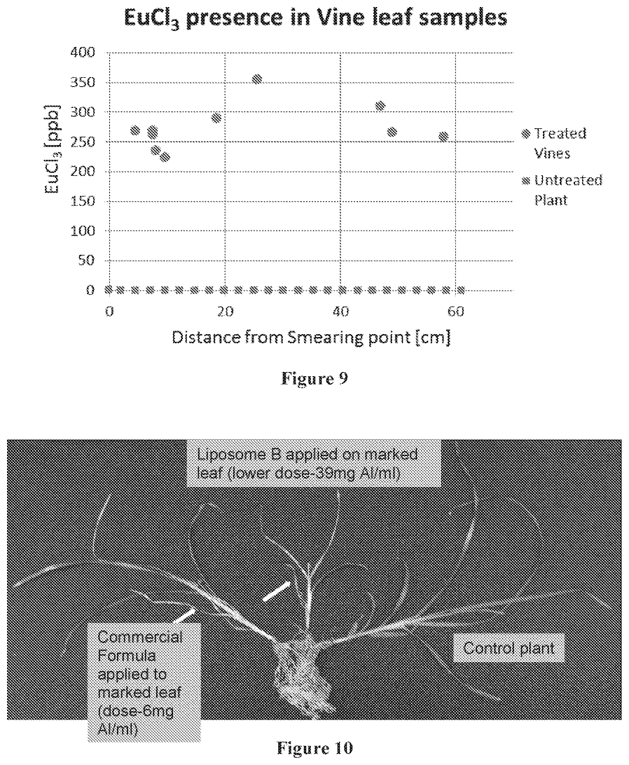

FIG. 10 is an image of the three plants (plants are not connected, the root systems are completely separate); showing that the liposomal formulation (Center Plant) was more active as a herbicide (less rejuvenation as compared to treatment with the commercial product) than the commercial product (Left Plant). In addition, the encapsulation within liposomes allowed reduction in the required administered dose.

In a similar experiment, a comparison was made between commercial and liposomal formulation of Glufosinate at 1/16 its recommended dose applied on the entire foliage of the plant by sparying.

Specifically, liposomal glufosinate was prepared, with a final glufosinate concentration in the liposomes of 0.35 mg/ml. Three treatment groups (in three replicas) were examined as follows:

Plant A: 1 ml per plant of commercial Glufosinate at a dose of 0.375 mg/ml.

Plant B: 1 ml per plant of Liposome B encapsulating Glufosinate at a dose of 0.35 mg/ml

Plant C: Untreated control (UTC)

FIG. 11A shows that after 22 days the plant treated with the commercial Glufosinate still had viable parts performing photosynthesis (centered plant) while the plant treated with liposomal glufosinate showed advanced necrosis in all parts (right end plant). This finding supports the advantage of delivering active agents not only in term of distribution, but also the fact that delivery by liposomes allows the reduction of dose required in order to obtain the desired effect (and as compared to commercial products).

The same trend continues 35 days after application. Plants treated with commercial formulation (left) continued to grow, while plants applied with liposome B (right) died as shown in FIG. 11B.

### Example 4—Delivery of Plant Nutrients

### Delivery of Mg or Mg+Fe

Tomato plants were grown under Mg-deficient conditions (interveinal chlorosis and leaf epinasty were observed). Two treatments were applied on the topmost leaf of the Mg-deficient tomato plant: Commercial unformulated MgSO₄ and Liposome A containing MgSO₄. Basipetal movement of Mg was evaluated through deficiency correction of lower leaves (3^{rd} and 4^{th} leaves) 10 days after application.

As shown in FIGS. 12A-12F, when applied to the topmost leaf, foliar liposomal MgSO₄ corrected deficiency (FIG. 12C, FIG. 12F) better than the effect of the commercial unformulated MgSO₄ (FIG. 12B, FIG. 12E), this being in comparison with non-treated plant, where chlorosis and necrosis were observed (FIG. 12A, FIG. 12D).

This observation supports the finding that liposomal formulation improved basipetal movement of Mg.

In a further experiment, Liposome A containing MgSO₄ and Liposome A containing Sequestrene (Fe-chelate) were applied on the topmost leaflet of Mg and Fe deficient tomato plants. Also in this case, basipetal movement was evaluated through deficiency (interveinal chlorosis, leaf epinasty) correction of lower leaves (3^{rd} and 4^{th} leaves) 10 days after application.

As shown in FIGS. 13A-13D, foliar application of liposomal Fe and Mg (encapsulated separately) by smearing corrected the deficiency (FIG. 13C, FIG. 13F) better than the commercial product (FIG. 13B, FIG. 13E) both as compared to non-treated group (FIG. 13A, FIG. 13D). The positive effect is evident from the better correction of intraveinal chlorosis and leaf epinasty of the plant treated with the liposomal formulation.

The same effect is observed in FIG. 13G, where the centered plant (liposome treated) exhibited the most pronounced growth.

Finally, acripetal movement of Fe applied on the lowest leaf by smearing was observed when Fe-deficient tomato plants were treated as follows:
Free Fe
Sequestrene™ (Fe-chelate)
Liposome A with Fe
Liposome A with Sequestrene™

The applied concentration was 50% of the recommended Fe rate for tomatoes. Acripetal movement was evaluated through deficiency correction of other parts of the foliage 10 days after application.

FIGS. 14A-14B show that foliar application of Fe (FIG. 14D) or Sequestrene (FIG. 14B) in liposomes to the lowest leaf of the plant corrects deficiency better, exhibited by the greater growth of the plants, indicating better acripetal movement of the liposomal formulations.

1.-36. (canceled)

37. A formulation comprising:
(i) liposomes comprising a lipid membrane and an intraliposomal aqueous core,
wherein,
the liposome has a diameter in the range of between 100 nm to 300 nm
the lipid membrane comprises two or more phospholipids, and non-liposome forming lipids comprising at least one plant derived sterol and at least one saponin,
at least one of said two or more phospholipids is a liposome forming lipid,
at least one of said two or more phospholipids has an unsaturated lipid tail; and
said lipid membrane comprises a mole % ratio between the said two or more phospholipids and the said at least one plant derived sterol and at least one saponin of between 20 mol %::80 mol % to 80 mol %::20 mol %;
and
(ii) an agriculturally acceptable carrier.

38. The formulation of claim 37, wherein said lipid membrane comprise one or a combination of phosphatidylcholine carrying at least one hydrocarbon chain selected from the group consisting of C16:0, C18:0, C18:1, C18:2, and C18:3.

39. The formulation of claim 37, wherein said lipid membrane comprises at least one unsaturated C16 or C18 phosphatidylcholine.

40. The formulation of claim 37, wherein said at least one of said lipids comprise a polar inositol head group.

41. The formulation of claim 37, wherein said liposome forming lipid comprising the acidic head group is selected from phosphatidylserine, phosphatidylglycerol, phosphatidic acid.

42. The formulation of claim 37, wherein said lipid membrane comprises one or more sterols being a phytosterol selected from the group consisting of β-sitosterol, stigmasterol, and ergosterol.

43. The formulation of claim 37, wherein said lipid membrane comprises a combination of liposome forming lipids comprising (i) saturated or unsaturated phosphatidylcholine, phosphatidylinositol and phosphatidic acid; and (ii) sterol.

44. The formulation of claim 37, wherein said lipid membrane comprises a positively charged lipid.

45. The formulation of claim 37, wherein said lipid membrane comprise up to 5% lipopolymer.

46. The formulation of claim 37, wherein said lipid membrane comprises a combination of (a) phospholipids comprising phosphatidylcholine (PC), phosphatidylinositol (P1), phosphatidic acid (PA), (b) sterols and (c) saponins.

47. A method of treating a plant comprising applying to a surface of a plant part a formulation comprising
(iii) liposomes comprising a lipid membrane and an intraliposomal aqueous core,
wherein,
the liposome has a diameter in the range of between 100 nm to 300 nm
the lipid membrane comprises two or more phospholipids, and non-liposome forming lipids comprising at least one plant derived sterol and at least one saponin,
at least one of said two or more phospholipids is a liposome forming lipid,
at least one of said two or more phospholipids has an unsaturated lipid tail; and
said lipid membrane comprises a mole % ratio between the said two or more phospholipids and the said at least one plant derived sterol and at least one saponin of between 20 mol %:80 mol % to 80 mol %:20 mol %;
and
(iv) an agriculturally acceptable carrier.

48. The method of claim 47, wherein said lipid membrane in the formulation comprises one or a combination of phosphatidylcholine carrying at least one hydrocarbon chain selected from the group consisting of C16:0, C18:0, C18:1, C18:2, and C18:3.

49. The method of claim 47, wherein said lipid membrane comprises at least one unsaturated C16 or C18 phosphatidylcholine.

50. The method of claim 47, wherein said at least one of said lipids comprise a polar inositol head group.

51. The method of claim 47, wherein said liposome forming lipid comprising the acidic head group is selected from phosphatidylserine, phosphatidylglycerol, phosphatidic acid.

52. The method of claim 47, wherein said lipid membrane in said formulation comprises up to 5% lipopolymer.

53. The method of claim 47, wherein said lipid membrane comprises a combination of (a) phospholipids comprising phosphatidylcholine (PC), phosphatidylinositol (PI), phosphatidic acid (PA), (b) sterols and (c) saponins.

54. The method of claim 47, wherein said formulation encapsulates in the intraliposomal core of the liposomes an agriculturally active agent, the amount of said agent in the liposome being effective to treat said plant.

55. An agricultural method comprising applying to soil a formulation comprising
(i) liposomes comprising a lipid membrane and an intraliposomal aqueous core,
wherein,
the liposome has a diameter in the range of between 100 nm to 300 nm
the lipid membrane comprises two or more phospholipids, and non-liposome forming lipids comprising at least one plant derived sterol and at least one saponin,
at least one of said two or more phospholipids is a liposome forming lipid,
at least one of said two or more phospholipids has an unsaturated lipid tail; and
said lipid membrane comprises a mole % ratio between the said two or more phospholipids and the said at least one plant derived sterol and at least one saponin of between 20 mol %:80 mol % to 80 mol %:20 mol %;
and
(ii) an agriculturally acceptable carrier.
